(19) United States
(12) Patent Application Publication
(10) Pub. No.: US 2023/0233451 A1
(43) Pub. Date: Jul. 27, 2023

(54) OSMOTIC DRUG DELIVERY IMPLANTS

(71) Applicant: LYRA THERAPEUTICS, INC., Watertown, MA (US)

(72) Inventors: Maria Palasis, Wellesley, MA (US); Danny Concagh, Medfield, MA (US); Changcheng You, Northbridge, MA (US)

(21) Appl. No.: 18/127,214

(22) Filed: Mar. 28, 2023

Related U.S. Application Data
(63) Continuation of application No. PCT/US2021/052331, filed on Sep. 28, 2021.

(51) Int. Cl. A61K 9/00; A61K 31/58

(52) U.S. Cl. CPC A61K 9/0004; A61K 9/0043; A61K 31/58

(57) ABSTRACT

The present invention relates to implantable devices, as well as their methods of use and manufacturing, having osmotic capabilities. Exemplary embodiments of the present invention include fiber and sheet based implantable devices for drug delivery to bodily lumens.

### FIELD OF INVENTION

The present invention is related to the fields of drug delivery and implantable devices. Devices, systems, and methods for use of osmotic drug delivery implants are contemplated herein.

### BACKGROUND

There is a demand for delivery systems that can sustainably deliver drug in a site-specific manner. Various drug delivery systems have been developed to optimize the therapeutic properties of drug products rendering them more safe, effective, reliable, and reducing issues of drug non-compliance. Implantable drug delivery systems are an example of such systems available for therapeutic use. Conventional biodegradable and nonbiodegradable (or biodurable) implants are available as monolithic systems or reservoir systems. The release kinetics of drugs from these implants depend on both the solubility and diffusion coefficient of the drug in the carrier polymer, the drug load, as well as the in vivo degradation rate of the carrier polymer, in the case of a biodegradable system. To offer this type of implant with more delivery versatility, the benefits of osmotic pumps have been integrated into biodegradable and nonbiodegradable/biodurable implants to create implants that deliver drug osmotically in conjunction with other release kinetics. These systems can deliver various types of active pharmaceutical ingredients (APIs), hydrophilic/lipophilic or small molecule/biomacromolecule, at steady rates.

### SUMMARY OF THE INVENTION

The present invention relates to implantable devices, as well as their methods of use and manufacturing, having osmotic capabilities. The present invention may be considered a further development of the embodiments disclosed in International Patent Publication WO2018195484A1, incorporated by reference in its entirety herein.

One exemplary embodiment of the present implantable devices is a device comprising one or more fibers, at least one of which is a permeable, hollow fiber comprising an active ingredient. This device, or scaffold, is not limited to the number of fibers or the structure the fibers take. Another exemplary embodiment of the present implantable devices is a device comprising a permeable, non-permeable or semi-permeable sheet which contains an active ingredient. The fiber or sheet may be considered a permeable, non-permeable or semi-permeable membrane.

The exemplary embodiment of the device comprises one or more fibers containing osmotic drug delivery components. In this embodiment, drug delivery components are comprised of one or more permeable, non-permeable or semi-permeable polymeric, hollow fibers containing a drug or active pharmaceutical ingredient (API) in the absence or presence of an osmogen. The present invention is not limited by the number or arrangement of the fiber(s). In one embodiment, fiber arrangement is a scaffold. In one embodiment, fiber arrangement is a spiral. In another embodiment, the fiber arrangement is braided. In this design, the implants comprise a fiber-based braid structure with multiple strands (e.g. 2 to 64), where at least one fiber comprises semi-permeable membrane that encapsulates the drug(s) or API(s).

The embodiment of the device comprising a sheet may contain osmotic drug delivery components as seen in FIG. 5. The permeable or semi-permeable sheets may be implanted flat or in a rolled state. In the rolled embodiment, the rolled sheet comprises an internal lumen. In this exemplary embodiment, drug delivery components are comprised of a semi-permeable polymeric hollow sheet containing a drug or active pharmaceutical ingredient (API) in the absence or presence of an osmogen.

The implantable device may comprise a permeable, non-permeable or semi-permeable membrane, such as one or more fibers or a sheet. One embodiment, permeability to fluid is achieved through the use of permeable materials. In another embodiment, permeability is achieved through one or more delivery orifices on the hollow fiber or sheet wall. Any number of orifices is contemplated, including, but not limited to, one, two, three, four, five, six, seven, eight, nine, ten, twenty-five, fifty, one hundred, two hundred, a thousand, etc. A non-permeable embodiment is contemplated, for example, a metal tube with holes, wherein said holes may be drilled.

In one embodiment, the devices herein may be coated or covered. It is not intended for the present invention to be limited by the type, such as an elastomer, the thickness, or degree of coverage (e.g. partial or complete) of the coating. The device may be completely or partially coated. In one embodiment, there may be an elastomer coating on the top of the permeable or semi-permeable membrane, such as on the hollow fibers or sheet, covering or not covering any delivery orifices. Elastomers may be coated onto the implants to provide them with self-expandability. One or more orifices may be formed on the semi-permeable membrane either before or after the elastomer coating. Coatings may range, for example, from between about 1 µm to about 25 µm in thickness (e.g., ranging from about 1 to 2 to 5 to 20 to 25 µm in thickness), among other possibilities. Coating thicknesses may also be less than 1 µm or greater than 25 µm.

In one embodiment, the device may be expandable. In one embodiment, the device may be self-expanding. In one embodiment, the device may be balloon-expandable. The many embodiments of the present disclosure may be self-expanding in that they are manufactured at a first diameter, subsequently reduced or “crimped” to a second, reduced diameter for placement within a delivery system, and self-expand towards the first diameter when extruded from the delivery catheter at an implantation site. The first diameter may be at least 10% larger than the diameter of the bodily cavity into which it is implanted in some embodiments. The scaffold may be designed to recover at least about 70%, at least about 80%, at least about 90%, up to about 100% of its manufactured, first diameter, in some embodiments.

In one embodiment, the device may be biodegradable or biodurable.

In one embodiment, various components of the device may be hydrophilic, hydrophobic, lipophilic, etc.

Upon implantation, a fluid, such as water, enters the device lumen through the permeable or semi-permeable wall, forming an osmotic pressure gradient that pushes the active pharmaceutical ingredient (API) out of the delivery orifices at a steady rate. These osmotic dosage forms function by allowing a fluid, such as water, around the implant to flow through the semi-permeable membrane, dissolve the API in the core or lumen so it can be released through the ports in the membrane by the osmotic pressure.

The present devices and systems may be used with a large multitude of active ingredients. Agents, or active ingredients, such as drugs or active pharmaceutical ingredients (APIs), may be embedded in porous or semi-porous fiber strands or sandwiched in porous or semi-porous sheets. In one embodiment, the agent is an active pharmaceutical ingredient. In one embodiment the present agent is a therapeutic agent. In one embodiment, the present agent is a glucocorticoid. In one embodiment, the present agent is mometasone furoate.

The active ingredients that may be used in this system include, but are not limited to, anticholinergic agents, antihistamines, anti-infective agents, anti-inflammatory agents, anti-scarring or antiproliferative agents, chemotherapeutic/antineoplastic agents, cytokines such as interferon and interleukins, decongestants, healing promotion agents and vitamins (e.g., retinoic acid, vitamin A, and their derivatives), hyperosmolar agents, immunomodulator/immunosuppressive agents, leukotriene modifiers, mucolytics, narcotic analgesics, small molecules, tyrosine kinase inhibitors, peptides, proteins, nucleic acids, vasoconstrictors, or combinations thereof. Anti-sense nucleic acid oligomers or other direct transactivation and/or transrepression modifiers of mRNA expression, transcription, and protein production may also be used. Anti-infective agents generally include antibacterial agents, antifungal agents, antiparasitic agents, antiviral agents, and antiseptics. Anti-inflammatory agents generally include steroidal and nonsteroidal anti-inflammatory agents.

Examples of antibacterial agents that may be suitable for use with the described systems include, but are not limited to, aminoglycosides, amphenicols, ansamycins, β-lactams (such as carbacephems, carbapenems, cephalosporins, cephamycins, monobactams, oxacephems, penicillins, and any of their derivatives), lincosamides, macrolides, nitrofurans, quinolones, sulfonamides, sulfones, tetracyclines, vancomycin, and any of their derivatives, or combinations thereof.

Examples of antifungal agents suitable for use with the described systems include, but are not limited to, allylamines, imidazoles, polyenes, thiocarbamates, triazoles, and any of their derivatives. Antiparasitic agents that may be employed include such agents as atovaquone, clindamycin, dapsone, iodoquinol, metronidazole, pentamidine, primaquine, pyrimethamine, sulfadiazine, trimethoprim/sulfamethoxazole, trimetrexate, and combinations thereof.

Examples of antiviral agents suitable for use with the described systems include, but are not limited to, acyclovir, famciclovir, valacyclovir, edoxudine, ganciclovir, foscamet, cidovir (vistide), vitrasert, formivirsen, HPMPA (9-(3-hydroxy-2-phosphonomethoxypropyl)adenine), PMEA (9-(2-phosphonomethoxyethyl)adenine), HPMPG (9-(3-Hydroxy-2-(Phosphonomet-hoxy)propyl)guanine), PMEG (9-[2-(phosphonomethoxy)ethyl]guanine), HPMPC (1-(2-phosphonomethoxy-3-hydroxypropyl)-cytosine), ribavirin, EICAR (5-ethynyl-1-beta-D-ribofuranosylimidazole-4-carboxamine), pyrazofurin (3-[beta-D-ribofuranosyl]-4-hydroxypyrazole-5-carboxamine), 3-Deazaguanine, GR-92938X (1-beta-D-ribofuranosylpyrazole-3,4-dicarboxami-de), LY253963 (1,3,4-thiadiazol-2-yl-cyanamide), RD3-0028 (1,4-dihydro-2,3-Benzodithiin), CL387626 (4,4′-bis[4,6-d][3-aminophenyl-N—, N-bis(2-carbamoylethyl)-sulfonilimino]-1,3,5-triazin-2-ylamino-biphenyl-2-,2′-disulfonic acid disodium salt), BABIM (Bis[5-Amidino-2-benzimidazoly-1]-methane), NIH351, and combinations thereof.

Examples of steroidal anti-inflammatory agents that may be used in the systems include 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, any of their derivatives, and combinations thereof. In one variation, the steroidal anti-inflammatory agent may be mometasone furoate. In another variation, fluticasone propionate may be included in the systems as the steroidal anti-inflammatory agent.

Suitable nonsteroidal anti-inflammatory agents include, but are not limited to, COX inhibitors (COX-1 or COX nonspecific inhibitors) (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam) and alkanones such as nabumetone) and selective COX-2 inhibitors (e.g., diaryl-substituted furanones such as rofecoxib; diaryl-substituted pyrazoles such as celecoxib; indole acetic acids such as etodolac and sulfonanilides such as nimesulide).

The chemotherapeutic/antineoplastic agents that may be used in the systems include, but are not limited to antitumor agents (e.g., cancer chemotherapeutic agents, biological response modifiers, vascularization inhibitors, hormone receptor blockers, cryotherapeutic agents or other agents that destroy or inhibit neoplasia or tumorigenesis) such as alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6 mercaptopurine and 5-fluorouracil (5FU), antitumor antibiotics and other compounds that act by binding or intercalating DNA and preventing RNA synthesis (e.g., doxorubicin, daunorubicin, epirubicin, idarubicin, mitomycin-C and bleomycin) plant (vinca) alkaloids and other anti-tumor agents derived from plants (e.g., vincristine and vinblastine), steroid hormones, hormone inhibitors, hormone receptor antagonists and other agents which affect the growth of hormone-responsive cancers (e.g., tamoxifen, herceptin, aromatase ingibitors such as aminoglutethamide and formestane, triazole inhibitors such as letrozole and anastrazole, steroidal inhibitors such as exemestane), antiangiogenic proteins, small molecules, gene therapies and/or other agents that inhibit angiogenesis or vascularization of tumors (e.g., meth-1, meth-2, thalidomide), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAI), combretastatin A4 prodrug (CA4P), SU6668, SU11248, BMS-275291, COL-3, EMD 121974, IMC-1C11, IM862, TNP-470, celecoxib (Celebrex), rofecoxib (Vioxx), interferon alpha, interleukin-12 (IL-12) or any of the compounds identified in Science Vol. 289, Pages 1197-1201 (Aug. 17, 2000), which is expressly incorporated herein by reference, biological response modifiers (e.g., interferon, bacillus calmette-guerin (BCG), monoclonal antibodies, interluken 2, granulocyte colony stimulating factor (GCSF), etc.), PGDF receptor antagonists, herceptin, asparaginase, busulphan, carboplatin, cisplatin, carmustine, chlorambucil, cytarabine, dacarbazine, etoposide, flucarbazine, fluorouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxol or paclitaxel, taxotere, analogs/congeners, derivatives of such compounds, and combinations thereof.

Examples of decongestants that may be incorporated in the systems include, but are not limited to, epinephrine, pseudoephedrine, oxymetazoline, phenylephrine, tetrahydrozolidine, and xylometazoline. Mucolytics that may be used include, but are not limited to, acetylcysteine, bromhexine, domase alpha, heparin, and guaifenesin.

It is not intended that the present implant be limited by the amount of active ingredient. The implant may comprise any amount of active ingredient, including, but not limited to about 1000 to about 10,000 micrograms. The present implant may comprise, 1000, 1500, 2000, 2500, 3000, 4000, 5000, 7500, 10,000 micrograms. In one embodiment, the implant comprises more than about 1000 micrograms. In one embodiment, the implant comprises more than about 2000 micrograms. In one embodiment, the implant comprises between about 1000 to about 5000 micrograms. In one embodiment, the implant comprises more than about 5000 micrograms.

The polymers used in the implants can be biodegradable, nonbiodegradable or biodurable. Polymers used in the implantable device include cellulose esters, alkyl-celluloses, and cellulose derivatives including methylcellulose, ethyl cellulose, hydroxyethyl cellulose, hydroxypropyl cellulose, hydroxylpropyl methyl cellulose, cellulose nitrate, cellulose acetate ethyl carbamate, cellulose acetate phthalate, cellulose acetate dimethaminoacetate, cellulose acetate ethyl carbonate, cellulose acetate chloroacetate, cellulose acetate ethyl oxalate, or any combination of any thereof. Synthetic polymers that may be used in the present device include partially and completely hydrolyzed alkylene-vinyl acetate copolymers, hydroxylated and unhydroxylated ethylene-vinyl acetate copolymers, derivatives of polystyrene such as poly(sodium styrenesulfonate) and poly(vinylbenzyltrimethylammonium chloride), homo- and copolymers of polyvinyl acetate, polymers of acrylic acid and methacrylic acid, copolymers of an alkylene oxide and alkyl glycidyl ether, polyurethanes, polyamide, polyshulphones, crosslinked polyethylene oxide), poly(alkylenes), poly(vinyl imidazole). Semi-permeable bioresorbable polymers that may be used in the present device include polyglycolic acid, polylactic acid, polycaprolactone, polydioxanone, poly(trimethylene carbonate), poly(3-hydroxybutyrate), poly(propiolactone), poly(ethylene succinate), poly(butylenes succinate), poly(3-hydroxybutyrate-co-3-hydroxyvalerate), poly(ester carbonate), poly(glycerol sebacate), and their copolymers and derivatives thereof.

Various osmogens, or osmotic agents, can be used to tailor, tune, define, adjust or change the osmotic pressure inside the semi-permeable membrane and consequently the release rate of the agent or active ingredient. These osmogens include but are not limited to sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. The osmogen can also be a water-soluble organic polymer such as hydroxy propyl methyl cellulose (HPMC), sodium carboxy methyl cellulose (Na CMC), polyethylene oxide (PEO), polyvinyl pyrrolidine (PVP), and methyl cellulose (MC) or a water-soluble amino acid such as alanine, glycine, leucine, and methionine. The osmotic agent may be the active pharmaceutical agent (API).In one embodiment, the device comprises a permeability enhancer. The permeability enhancer serves to facilitate the permeability of the drug into tissues or across tissue boundaries, such as the blood brain barrier. In one embodiment, the device comprises a permeability enhancer in addition to one or more therapeutic agents, osmotic agents, and other aiding agents (wicking agents, surfactants, swelling agents, solubilizing agents, etc.) Examples of permeability enhancers include Poly Vinyl Pyrrolidine, Carboxymethyl Cellulose, mucosal tissue permeability enhancers such as laurocapram, dimethylacetamide (DMAC), n-methyl-2-pyrrolidone, polyarginine, glycols (e.g., diethylene glycol, tetraethylene glycol), lauric acid, oleic acid, polyxoyethyleen-2-oleyl ether, eucalyptus oil, menthol, 4-decyloxazolidin-2-one, and any combination thereof. Examples of blood brain barrier permeasility enhancers include leukotrienes, bradykinin agonists, histamine, tight junction disintegrants (e.g., zonulin, zotto), short chain alkyl glycerol (e.g., 1-O-pentylglycerol), and any combination thereof.

In some embodiments, the implant may comprise one or more swelling agents. In one embodiment, the device comprises a swelling agent in addition to one or more therapeutic agents, osmotic agents, permeability enhancers, and other aiding agents (wicking agents, surfactants, solubilizing agents, etc.) Examples of swelling agents include hydrophilic crosslinked polymers, hydrogels, carbopol, cellulose, starch, and any combination thereof. The swelling agent may be contained within a fiber, in one embodiment.

In some embodiments, the implant comprises one or more wicking agents. In one embodiment, the device comprises a wicking agent in addition to one or more therapeutic agents, osmotic agents, permeability enhancers and other aiding agents (surfactants, swelling agents, solubilizing agents, etc.) Examples of wicking agents include Polyesters, polyethylene, low molecular weight polyvinyl pyrrolidone (PVP), n-methyl-2-pyrrolidone, colloidal silicon dioxide, kaolin, titanium dioxide, bentonite, magnesium aluminum silicate, and any combination thereof. The wicking agent may be contained within a fiber, in one embodiment.

In some embodiments, the implant comprises one or more surfactants. In one embodiment, the device comprises a surfactant in addition to one or more therapeutic agents, osmotic agents, permeability enhancers, and other aiding agents (wicking agents, swelling agents, solubilizing agents, etc.) Examples of surfactants include Sodium lauryl sulphate, Sodium dodecyl sulphate, Polysorbate, Tween, pluronics, silicone surfactants, fluorosurfactants, and any combination thereof. The surfactant may be contained within a fiber, in one embodiment.

In some embodiments, the implant comprises one or more solubilizing agents. In one embodiment, the device comprises a solubilizing agent in addition to one or more therapeutic agents, osmotic agents, and other aiding agents (wicking agents, surfactants, swelling agents, etc.) Examples of solubilizing agents include Glycols, Cyclodextrins, mineral oils, and any combination thereof. The solubilizing agent may be contained within a fiber, in one example.

In some embodiments, a fiber comprises a single agent. In some embodiments, a fiber may comprise a plurality of agents. In some embodiments, a plurality of agents may be mixed homogenously in a single fiber. In some embodiments, a plurality of agents may be mixed heterogeneously in a single fiber, for example adjacent plugs of agent.

In one embodiment, different strands or fibers may contain different agents. In one embodiment, an implant may comprise plurality strands having different agents, such as one or more therapeutic agents, one or more osmogens, one or more permeability enhancers, one or more wicking agents, one or more surfactants, one or more swelling agents, or one or more solubilizing agents. In one embodiment an implant is contemplated, having one or more fibers comprising a therapeutic agent and one or more fibers comprising an agent selected from the group consisting of an osmogen, a permeability enhancer, a wicking agent, a surfactant, a swelling agent, and a solubilizing agent. In one embodiment, an implant may comprise a first portion of fibers comprising one or more agents and a second portion of fibers comprising one or more agents. In some embodiments, agents may be mixed homogenously in some fibers, but not in others.

In one embodiment, agents within a fiber may be continuous, and not intermittent or sporadic. In another embodiment, the agents within a fiber may be intermittent, such as in adjacent or non-adjacent plugs.

The present inventions are advantageous as they can be formed through a variety of manufacturing methods, such as coextrusion, filling, or successive coating. In one embodiment, drug-encapsulated fibers are formed by the coextrusion of the API(s) and the semi-permeable polymers into a core-shell structure. In another embodiment, drug-encapsulated fibers are formed by first creating hollow fibers comprising a lumen, followed by introducing the API(s) into the lumen. The lumen of the osmotic drug delivery fiber may comprise a variety of substances, such as the API, osmogens, permeability enhancers, and other aiding agents (e.g. wicking agents, surfactants, swelling agents, and solubilizing agents), while the shell is comprised of the semi-permeable polymer. In another embodiment, the drug-encapsulated fibers are formed by coating a solid polymer fiber core successively with the API(s) and a semi-permeable polymer membrane. The API is encapsulated in between the polymer fiber core and the semi-permeable polymer membrane to form a sandwich structure. The two ends of the fiber comprising API may be blocked through polymer coating or welding. Following the fabrication of the individual fibers, one or more fibers, some containing API and some not, can be formed into an implant or scaffold.

One or more fibers described above can be fabricated into spiral scaffolds, with at least one fiber comprising API. Following the blockage of the ends of the fibers, one or more drug delivery orifices may be placed on the semi-permeable wall using, for example, laser drilling. A shape memory polymer may be attached to the side or even serve as the core of the drug delivery fiber, to maintain the spiral shape of the fiber after implantation. In addition, elastomer can be coated onto the spiral scaffolds to enhance their recoverability post implantation.

Multi-stranded scaffolds comprising other fiber arrangements are manufactured following fabrication of single fibers, comprising at least one strand of those API-encapsulated fibers. The fibers may be arranged in a spiral, as stated above, or a braid, mesh, etc. The scaffolds can be conformally coated with an elastomer to provide the scaffold self-expandability. Following the blockage of the ends of the fibers, one or more drug delivery orifices may be placed on the semi-permeable wall using, for example, laser drilling.

Before or after such elastomer coating or arrangement of fibers, one or more delivery orifices are introduced onto the semi-permeable membrane of each API-encapsulated fiber through either mechanical drilling or laser drilling. Either luminal or abluminal delivery orifices can be formed accordingly. The size, density, and location of the delivery orifices are determined by the API used, the target implantation sites, and the dosing requirement. Furthermore, the delivery orifices can also be formed by a salt-leaching approach, where inorganic salt granules are present during the semi-permeable membrane formation. Upon implantation, the salt will dissolve and leach out to form drug delivery orifices in situ. The number and size of the orifices can be tuned by tailoring the size and quantity of salt granules within the membrane.

The sheet embodiment also may be manufactured in a variety of methods. APIs and aiding agents are encapsulated in between two polymer membranes to form a drug release sheet. One or both polymer membranes are semi-permeable membranes. Drug delivery orifices can be drilled on either polymer membranes to allow drug release. Optional elastomer coating can be further introduced onto the rolled sheets to improve their self-expandability.

Systems are also contemplated comprising an implant having (i) a membrane wall containing at least one orifice, (ii) a lumen loaded with an agent, and (iii) a coating at least partially covering said membrane wall, wherein said implant is configured to have a release rate that allows for release of agent for more than 12 weeks, release of 20 to 80% of said agent during the first 12 weeks, a substantially linear release of said agent between 1 and 12 weeks, and/or a substantially linear release of said agent that is exhibited in vitro in pH 7.4 PBS buffer containing 2% SDS at 37° C. International Patent Publication WO2018195484A1 is incorporated by reference in its entirety herein, and details release of agents from implant embodiments discussed herein.

In one embodiment, an implant is contemplated comprising (i) a membrane wall containing at least one orifice, (ii) a lumen containing an agent, and (iii) a coating at least partially covering said membrane wall. In one embodiment, said lumen further comprises an osmogen. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said lumen further comprises a permeability enhancer. In one embodiment, said lumen further comprises at least one aiding agent selected from the group consisting of a wicking agent, a swelling agent, a surfactant, and a solubilizing agent. In one embodiment, said coating is elastomeric. In one embodiment, said coating is bioresorbable. In one embodiment, said coating is biodurable. In one embodiment, said membrane wall is water permeable. In one embodiment, said membrane wall is not permeable to said agent. In one embodiment, said agent is a therapeutic agent. In one embodiment, said agent is a drug. In one embodiment, said agent is a steroid. In one embodiment, said agent is a glucocorticoid. In one embodiment, said agent is mometasone furoate. In one embodiment, said agent is a solid. In one embodiment, said agent is continuous throughout said lumen. In one embodiment, said agent is a hydrophilic or lipophilic molecule. In one embodiment, said agent is a small molecule or biomacromolecule. In one embodiment, said coating is covering said at least one orifice. In one embodiment, said coating is not covering said at least one orifice. In one embodiment, said implant further comprises a topcoat at least partially covering said coating. In one embodiment, said lumen further comprises a polymer core. In one embodiment, said agent is contained between said polymer core and said membrane wall. In one embodiment, said polymer is a shape-memory polymer. In one embodiment, said polymer is swellable. In one embodiment, said polymer is loosely crosslinked. In one embodiment, said implant is expandable. In one embodiment, said implant is self-expanding. In one embodiment, said implant is balloon expandable. In one embodiment, said implant is configured to be delivered to a mucosal tissue. In one embodiment, said mucosal tissue is tissue of the middle meatus. In one embodiment, said mucosal tissue is tissue of the olfactory cleft. In one embodiment, said implant is configured to be delivered to a nasal cavity. In one embodiment, said nasal cavity is the middle meatus. In one embodiment, said nasal cavity is the olfactory cleft. In one embodiment, said implant is non-metallic. In one embodiment, said implant is configured to be administered without a needle. In one embodiment, said implant lacks a retention frame.

In one embodiment, an implant is contemplated comprising (i) a membrane wall containing at least one orifice, (ii) a lumen containing an agent and an osmogen. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, wherein said osmogen is a water-soluble amino acid. In one embodiment, wherein said osmogen is selected from the group consisting of alanine, glycine, leucine, methionine, and their combinations at any ratios. In one embodiment, said lumen further comprises a permeability enhancer. In one embodiment, said lumen further comprises at least one aiding agent selected from the group consisting of a wicking agent, a surfactant, a swelling agent, and a solubilizing agent. In one embodiment, said implant further comprises (iii) a coating at least partially covering said membrane wall. In one embodiment, said coating is elastomeric. In one embodiment, said coating is bioresorbable. In one embodiment, said coating is biodurable. In one embodiment, said membrane wall is water permeable. In one embodiment, said membrane wall is not permeable to said agent. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is continuous throughout said lumen. In one embodiment, said agent is a hydrophilic or lipophilic molecule. In one embodiment, said agent is a small molecule or biomacromolecule. In one embodiment, said coating is covering said at least one orifice. In one embodiment, said coating is not covering said at least one orifice. In one embodiment, said devices further comprises a topcoat at least partially coating said coating. In one embodiment, said lumen further comprises a polymer core. In one embodiment, agent is contained between said polymer core and said membrane wall. In one embodiment, said polymer is a shape-memory polymer. In one embodiment, said polymer is swellable. In one embodiment, said polymer is loosely crosslinked. In one embodiment, said implant is expandable. In one embodiment, said implant is self-expanding. In one embodiment, said implant is balloon expandable. In one embodiment, said implant is configured to be delivered to a mucosal tissue. In one embodiment, said mucosal tissue is tissue of the middle meatus. In one embodiment, said mucosal tissue is tissue of the olfactory cleft. In one embodiment, said implant is configured to be delivered to a nasal cavity. In one embodiment, said nasal cavity is the middle meatus. In one embodiment, said nasal cavity is the olfactory cleft. In one embodiment, said implant is non-metallic. In one embodiment, said implant is configured to be administered without a needle. In one embodiment, said implant lacks a retention frame.

In one embodiment, an implant is contemplated comprising a plurality of fibers at least one of said fibers comprising (i) a membrane wall containing at least one orifice, (ii) a lumen containing an agent, and (iii) a coating at least partially covering said membrane wall. In one embodiment, said fibers are bioresorbable. In one embodiment, said coating is elastomeric. In one embodiment, said coating is biodurable. In one embodiment, said membrane wall is water permeable. In one embodiment, said membrane wall is not permeable to said agent. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is continuous throughout said lumen. In one embodiment, said agent is a hydrophilic or lipophilic molecule. In one embodiment, said agent is a small molecule or biomacromolecule. In one embodiment, said coating is covering said at least one orifice. In one embodiment, said coating is not covering said at least one orifice. In one embodiment, said implant further comprises a topcoat at least partially covering said coating. In one embodiment, said plurality of fibers are in an arrangement selected from the group consisting of a braid, a spiral, a mesh, and a weave. In one embodiment, said lumen further comprises an osmogen. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said lumen further comprises a permeability enhancer. In one embodiment, said lumen further comprises at least one aiding agent selected from the group consisting of a wicking agent, a surfactant, a swelling agent, and a solubilizing agent. In one embodiment, said coating is not covering said at least one orifice. In one embodiment, said lumen further comprises a polymer fiber core. In one embodiment, said agent is contained between said polymer fiber core and said membrane wall. In one embodiment, said polymer is a shape-memory polymer. In one embodiment, said polymer is swellable. In one embodiment, said polymer is loosely crosslinked. In one embodiment, said implant is expandable. In one embodiment, said implant is balloon expandable. In one embodiment, said implant is self-expanding. In one embodiment, said implant is configured to be delivered to a mucosal tissue. In one embodiment, said mucosal tissue is tissue of the middle meatus. In one embodiment, said mucosal tissue is tissue of the olfactory cleft. In one embodiment, said implant is configured to be delivered to a nasal cavity. In one embodiment, said nasal cavity is the middle meatus. In one embodiment, said nasal cavity is the olfactory cleft. In one embodiment, said implant is non-metallic. In one embodiment, said implant is configured to be administered without a needle. In one embodiment, said implant lacks a retention frame. In one embodiment, said at least one fiber has a diameter between 100-500 µm.

The present invention in one embodiment contemplates an implant comprising a plurality of fibers at least one of said fibers comprising (i) a membrane wall containing at least one orifice, (ii) a lumen containing an agent and an osmogen, and (iii) a coating at least partially covering said membrane wall. In one embodiment, said fibers are bioresorbable. In one embodiment, said coating is elastomeric. In one embodiment, said coating is biodurable. In one embodiment, said membrane wall is water permeable. In one embodiment, said membrane wall is not permeable to said agent. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is continuous throughout said lumen. In one embodiment, said agent is a hydrophilic or lipophilic molecule. In one embodiment, said agent is a small molecule or biomacromolecule. In one embodiment, said coating is covering said at least one orifice. In one embodiment, said coating is not covering said at least one orifice. In one embodiment, wherein said implant further comprises a topcoat at least partially covering said coating. In one embodiment, said plurality of fibers are in an arrangement selected from the group consisting of a braid, a spiral, a mesh, and a weave. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said lumen further comprises a permeability enhancer. In one embodiment, said lumen further comprises at least one aiding agent selected from the group consisting of a wicking agent, a surfactant, a swelling agent, and a solubilizing agent. In one embodiment, said coating is not covering said at least one orifice. In one embodiment, said lumen further comprises a polymer fiber core. In one embodiment, agent is contained between said polymer fiber core and said membrane wall. In one embodiment, said polymer is a shape-memory polymer. In one embodiment, said polymer is swellable. In one embodiment, said polymer is loosely crosslinked. In one embodiment, said implant is expandable. In one embodiment, said implant is balloon expandable. In one embodiment, said implant is self-expanding. In one embodiment, said implant is configured to be delivered to a mucosal tissue. In one embodiment, said mucosal tissue is tissue of the middle meatus. In one embodiment, said mucosal tissue is tissue of the olfactory cleft. In one embodiment, said implant is configured to be delivered to a nasal cavity. In one embodiment, said nasal cavity is the middle meatus. In one embodiment, said nasal cavity is the olfactory cleft. In one embodiment, said implant is non-metallic. In one embodiment, said implant is configured to be administered without a needle. In one embodiment, said implant lacks a retention frame. In one embodiment, said at least one fiber has a diameter between 100-500 µm.

The present invention in one embodiment contemplates an implant comprising at least one spiral fiber having (i) a membrane wall containing at least one orifice, (ii) a lumen containing an agent, and (iii) a coating at least partially covering said membrane wall. In one embodiment, said at least one spiral fiber is bioresorbable. In one embodiment, said at least one spiral fiber is biodurable. In one embodiment, said membrane wall is water permeable. In one embodiment, said membrane wall is not permeable to said agent. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is continuous throughout said lumen. In one embodiment, said agent is a hydrophilic or lipophilic molecule. In one embodiment, said agent is a small molecule or biomacromolecule. In one embodiment, said coating is elastomeric. In one embodiment, said coating is bioresorbable. In one embodiment, said coating is biodurable. In one embodiment, said coating is covering said at least one orifice. In one embodiment, said coating is not covering said at least one orifice. In one embodiment, wherein said implant further comprises a topcoat at least partially covering said coating. In one embodiment, said lumen further comprises an osmogen. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said lumen further comprises a permeability enhancer. In one embodiment, said lumen further comprises at least one aiding agent selected from the group consisting of a wicking agent, a surfactant, a swelling agent, and a solubilizing agent. In one embodiment, said lumen further comprises a polymer fiber core. In one embodiment, agent is contained between said polymer fiber core and said membrane wall. In one embodiment, said polymer is a shape-memory polymer. In one embodiment, said polymer is swellable. In one embodiment, said polymer is loosely crosslinked. In one embodiment, said implant is expandable. In one embodiment, said implant is self-expanding. In one embodiment, said implant is balloon expandable. In one embodiment, said implant is configured to be delivered to a mucosal tissue. In one embodiment, said mucosal tissue is tissue of the middle meatus. In one embodiment, said mucosal tissue is tissue of the olfactory cleft. In one embodiment, said implant is configured to be delivered to a nasal cavity. In one embodiment, said nasal cavity is the middle meatus. In one embodiment, said nasal cavity is the olfactory cleft. In one embodiment, said implant is non-metallic In one embodiment, said implant is configured to be administered without a needle. In one embodiment, said implant lacks a retention frame. In one embodiment, said fiber has a diameter between 100-500 µm.

The present invention in one embodiment contemplates an implant comprising at least one spiral fiber having (i) a membrane wall containing at least one orifice, (ii) a lumen containing an agent and an osmogen, and (iii) a coating at least partially covering said membrane wall. In one embodiment, said at least one spiral fiber is bioresorbable. In one embodiment, said at least one spiral fiber is biodurable. In one embodiment, said membrane wall is water permeable. In one embodiment, said membrane wall is not permeable to said agent. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is continuous throughout said lumen. In one embodiment, said agent is a hydrophilic or lipophilic molecule. In one embodiment, said agent is a small molecule or biomacromolecule. In one embodiment, said coating is elastomeric. In one embodiment, said coating is bioresorbable. In one embodiment, said coating is biodurable. In one embodiment, said coating is covering said at least one orifice. In one embodiment, said coating is not covering said at least one orifice. In one embodiment, wherein said implant further comprises a topcoat at least partially coating said coating. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said lumen further comprises a permeability enhancer. In one embodiment, said lumen further comprises at least one aiding agent selected from the group consisting of a wicking agent, a surfactant, a swelling agent, and a solubilizing agent. In one embodiment, said lumen further comprises a polymer fiber core. In one embodiment, said agent is contained between said polymer fiber core and said membrane wall. In one embodiment, said polymer is a shape-memory polymer. In one embodiment, said polymer is swellable. In one embodiment, said polymer is loosely crosslinked. In one embodiment, said implant is expandable. In one embodiment, said implant is self-expanding. In one embodiment, said implant is balloon expandable. In one embodiment, said implant is configured to be delivered to a mucosal tissue. In one embodiment, said mucosal tissue is tissue of the middle meatus. In one embodiment, said mucosal tissue is tissue of the olfactory cleft. In one embodiment, said implant is configured to be delivered to a nasal cavity. In one embodiment, said nasal cavity is the middle meatus. In one embodiment, said nasal cavity is the olfactory cleft. In one embodiment, said implant is non-metallic. In one embodiment, said implant is configured to be administered without a needle. In one embodiment, said implant lacks a retention frame. In one embodiment, said fiber has a diameter between 100-500 µm.

The present invention in one embodiment contemplates an implant comprising (i) a permeable outer membrane wall containing at least one orifice, (ii) a lumen containing an agent, and (iii) a permeable inner membrane wall. In one embodiment, said implant is tubular and further comprises a coating at least partially over said outer membrane wall. In one embodiment, said permeable inner membrane wall contains at least one orifice. In one embodiment, said implant further comprises an osmogen. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said coating is elastomeric. In one embodiment, said coating is bioresorbable. In one embodiment, said coating is biodurable. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is continuous throughout said lumen. In one embodiment, said agent is a hydrophilic or lipophilic molecule. In one embodiment, said agent is a small molecule or biomacromolecule. In one embodiment, said implant is expandable. In one embodiment, said implant is self-expanding. In one embodiment, said implant is balloon expandable. In one embodiment, said agent is contained between said outer membrane wall and said inner membrane wall. In one embodiment, said implant is configured to be delivered to a mucosal tissue. In one embodiment, said mucosal tissue is tissue of the middle meatus. In one embodiment, said mucosal tissue is tissue of the olfactory cleft. In one embodiment, said implant is configured to be delivered to a nasal cavity. In one embodiment, said nasal cavity is the middle meatus. In one embodiment, said nasal cavity is the olfactory cleft. In one embodiment, said implant is non-metallic. In one embodiment, said implant is configured to be administered without a needle. In one embodiment, said implant lacks a retention frame.

The present invention in one embodiment contemplates an implant comprising (i) a first membrane layer containing at least one orifice, (ii) a second membrane layer, and (iii) an active layer containing an agent and an osmogen disposed between said first and second membrane layers. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said implant further comprises a coating. In one embodiment, said coating is elastomeric. In one embodiment, said coating is bioresorbable. In one embodiment, said coating is biodurable. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is a hydrophilic or lipophilic molecule. In one embodiment, said agent is a small molecule or biomacromolecule. In one embodiment, said implant is configured to be delivered to a mucosal tissue. In one embodiment, said mucosal tissue is tissue of the middle meatus. In one embodiment, said mucosal tissue is tissue of the olfactory cleft. In one embodiment, said implant is configured to be delivered to a nasal cavity. In one embodiment, said nasal cavity is the middle meatus. In one embodiment, said nasal cavity is the olfactory cleft. In one embodiment, said implant is non-metallic. In one embodiment, said implant is configured to be administered without a needle. In one embodiment, said implant lacks a retention frame.

In one embodiment, an implant is contemplated comprising (i) a membrane wall containing at least one orifice, (ii) a lumen containing a permeability enhancer, and (iii) a coating at least partially covering said membrane wall. In one embodiment, said implant further comprises a therapeutic agent. In one embodiment, said implant further comprises an osmogen. In one embodiment, said implant further comprises at least one aiding agent selected from the group consisting wicking agents, surfactants, swelling agents, solubilizing agents, etc.

In one embodiment, an implant is contemplated comprising (i) a membrane wall containing at least one orifice, (ii) a lumen containing an agent and a permeability enhancer. In one embodiment, said agent is a therapeutic agent. In one embodiment, said agent is an osmogen. In one embodiment, said agent is selected from the group consisting wicking agents, surfactants, swelling agents, solubilizing agents, etc.

In one embodiment, an implant is contemplated comprising a plurality of fibers, wherein a first portion of said fibers comprise a first agent, and a second portion of said fibers comprise a second agent. In one embodiment, said first agent is selected from the group consisting of a therapeutic agent, an osmogen, a permeability enhancer, a wicking agent, a surfactant, a swelling agent, and a solubilizing agent. In one embodiment, said second agent is selected from the group consisting of a therapeutic agent, an osmogen, a permeability enhancer, a wicking agent, a surfactant, a swelling agent, and a solubilizing agent. In one embodiment, said implant further comprises a third portion of said fibers comprising a third agent. In one embodiment, said third agent is selected from the group consisting of a therapeutic agent, an osmogen, a permeability enhancer, a wicking agent, a surfactant, a swelling agent, and a solubilizing agent. In one embodiment, said implant further comprises a fourth portion of said fibers comprising a fourth agent. In one embodiment, said fourth agent is selected from the group consisting of a therapeutic agent, an osmogen, a permeability enhancer, a wicking agent, a surfactant, a swelling agent, and a solubilizing agent. In one embodiment, said implant further comprises a fifth portion of said fibers comprising a fifth agent. In one embodiment, said fifth agent is selected from the group consisting of a therapeutic agent, an osmogen, a permeability enhancer, a wicking agent, a surfactant, a swelling agent, and a solubilizing agent. In one embodiment, at least one of said fibers further comprise a coating. In one embodiment, said first portion of fibers comprise a third agent. In one embodiment, said second portion of fibers comprise a fourth agent.

In one embodiment, an implant is contemplated comprising a plurality of fibers, one or more of said fibers comprising (i) a first membrane wall containing at least one orifice, (ii) a first lumen containing a first agent, and one or more of said fibers comprising (i) a second membrane wall containing at least one orifice, and (ii) a second lumen containing a second agent. In one embodiment, said first agent is selected from the group consisting of a therapeutic agent, an osmogen, a permeability enhancer, a wicking agent, a surfactant, a swelling agent, and a solubilizing agent. In one embodiment, said second agent is selected from the group consisting of a therapeutic agent, an osmogen, a permeability enhancer, a wicking agent, a surfactant, a swelling agent, and a solubilizing agent. In one embodiment, one or more of said fibers comprise (i) a third membrane wall containing at least one orifice, and (ii) a third lumen containing a third agent. In one embodiment, said third agent is selected from the group consisting of a therapeutic agent, an osmogen, a permeability enhancer, a wicking agent, a surfactant, a swelling agent, and a solubilizing agent. In one embodiment, one or more of said fibers comprise (i) a fourth membrane wall containing at least one orifice, and (ii) a fourth lumen containing a fourth agent. In one embodiment, said fourth agent is selected from the group consisting of a therapeutic agent, an osmogen, a permeability enhancer, a wicking agent, a surfactant, a swelling agent, and a solubilizing agent. In one embodiment, one or more of said fibers comprise (i) a fifth membrane wall containing at least one orifice, and (ii) a fifth lumen containing a fifth agent. In one embodiment, said fifth agent is selected from the group consisting of a therapeutic agent, an osmogen, a permeability enhancer, a wicking agent, a surfactant, a swelling agent, and a solubilizing agent. In one embodiment, said fibers are bioresorbable. In one embodiment, at least one of said fibers comprises a coating. In one embodiment, said coating is elastomeric. In one embodiment, said coating is biodurable. In one embodiment, any of said membrane walls are water permeable. In one embodiment, any of said membrane walls are not permeable to said agent. In one embodiment, any of said agents are continuous throughout said lumen. In one embodiment, said first agent is a hydrophilic or lipophilic molecule. In one embodiment, said first agent is a small molecule or biomacromolecule. In one embodiment, said coating is covering said at least one orifice. In one embodiment, said coating is not covering said at least one orifice. In one embodiment, said implant further comprises a topcoat at least partially covering said coating. In one embodiment, said plurality of fibers are in an arrangement selected from the group consisting of a braid, a spiral, a mesh, and a weave. In one embodiment, said lumen further comprises an osmogen. In one embodiment, said osmogen is a salt or sugar. In one embodiment, said first agent is an osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said first agent is an osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, any of said lumens further comprises a polymer fiber core. In one embodiment, said agents are contained between said polymer fiber core and said membrane wall. In one embodiment, said polymer is a shape-memory polymer. In one embodiment, said polymer is swellable. In one embodiment, said polymer is loosely crosslinked. In one embodiment, said implant is expandable. In one embodiment, said implant is balloon expandable. In one embodiment, said implant is self-expanding. In one embodiment, said implant is configured to be delivered to a mucosal tissue. In one embodiment, said mucosal tissue is tissue of the middle meatus. In one embodiment, said mucosal tissue is tissue of the olfactory cleft. In one embodiment, said implant is configured to be delivered to a nasal cavity. In one embodiment, said nasal cavity is the middle meatus. In one embodiment, said nasal cavity is the olfactory cleft. In one embodiment, said implant is non-metallic. In one embodiment, said implant is configured to be administered without a needle. In one embodiment, said implant lacks a retention frame. In one embodiment, said at least one fiber has a diameter between 100-500 µm.

In one embodiment an implant is contemplated, having one or more fibers comprising a therapeutic agent and one or more fibers comprising an agent selected from the group consisting of an osmogen, a permeability enhancer, a wicking agent, a surfactant, a swelling agent, and a solubilizing agent. In one embodiment, at least one of said fibers further comprise a coating. In one embodiment, at least one of said fibers further comprise a coating. In one embodiment, at least a portion of said fibers further comprise at least one orifice.

In one embodiment, a method of delivering an agent is contemplated comprising: a) providing an implant comprising (i) a membrane wall containing at least one orifice, (ii) a lumen containing an agent, and (iii) a coating at least partially covering said membrane wall; and b) placing said implant in contact with a wet surface of a body cavity, such that said agent is delivered to said wet surface through osmosis. In one embodiment, said wet surface of a body cavity is within a human or animal body. In one embodiment, said lumen further comprises an osmogen. In one embodiment, said osmogen is a salt or sugar. In one embodiment, said osmogen is selected from the group comprising sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said coating is elastomeric. In one embodiment, said coating is bioresorbable. In one embodiment, said coating is biodurable. In one embodiment, said coating degrades upon contact with said wet surface. In one embodiment, said coating swells upon contact with said wet surface. In one embodiment, said membrane wall is water permeable. In one embodiment, said membrane wall is not permeable to said agent. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is continuous throughout said lumen. In one embodiment, said agent is a hydrophilic or lipophilic molecule. In one embodiment, said agent is a small molecule or biomacromolecule. In one embodiment, said elastomeric coating is covering said at least one orifice. In one embodiment, said elastomeric coating is not covering said at least one orifice. In one embodiment, said method further comprises a topcoat at least partially coating said elastomeric coating. In one embodiment, said lumen further comprises a polymer fiber core. In one embodiment, said agent is contained between said polymer fiber core and said membrane wall. In one embodiment, said polymer is a shape-memory polymer fiber. In one embodiment, said polymer is swellable. In one embodiment, said polymer is loosely crosslinked. In one embodiment, said polymer swells such that said agent is delivered to said wet surface through osmosis. In one embodiment, said implant is expandable. In one embodiment, said implant is self-expanding. In one embodiment, said implant is balloon expandable. In one embodiment, said wet surface is the surface of mucosal tissue. In one embodiment, said mucosal tissue is tissue of the middle meatus. In one embodiment, said mucosal tissue is tissue of the olfactory cleft. In one embodiment, said body cavity is a nasal cavity. In one embodiment, said body cavity is the middle meatus. In one embodiment, said body cavity is the olfactory cleft. In one embodiment, said implant is non-metallic. In one embodiment, said implant is placed in contact with a wet surface of a body cavity without a needle. In one embodiment, said implant lacks a retention frame.

In one embodiment, a method of delivering an agent comprising: a) providing an implant comprising (i) a membrane wall containing at least one orifice, (ii) a lumen containing an agent and an osmogen, and (iii) a coating at least partially covering said membrane wall; and b) placing said implant in contact with a wet surface of a body cavity, such that said agent is delivered to said wet surface through osmosis. In one embodiment, said wet surface of a body cavity is within a human or animal body. In one embodiment, said osmogen is a salt or sugar. In one embodiment, said osmogen is selected from the group comprising sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said coating is elastomeric. In one embodiment, said coating is bioresorbable. In one embodiment, said coating is biodurable. In one embodiment, said method further comprises the step of the coating degrading. In one embodiment, said membrane wall is water permeable. In one embodiment, said membrane wall is not permeable to said agent. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is continuous throughout said lumen. In one embodiment, said agent is a hydrophilic or lipophilic molecule. In one embodiment, said agent is a small molecule or biomacromolecule. In one embodiment, said coating is covering said at least one orifice. In one embodiment, said coating is not covering said at least one orifice. In one embodiment, said method further comprises a topcoat at least partially covering said coating. In one embodiment, said lumen further comprises a polymer core. In one embodiment, said agent is contained between said polymer core and said membrane wall. In one embodiment, said polymer is a shape-memory polymer. In one embodiment, said polymer is swellable. In one embodiment, said polymer is loosely crosslinked. In one embodiment, said implant is expandable. In one embodiment, said implant is self-expanding. In one embodiment, said implant is balloon expandable. In one embodiment, said wet surface is the surface of mucosal tissue. In one embodiment, said mucosal tissue is tissue of the middle meatus. In one embodiment, said mucosal tissue is tissue of the olfactory cleft. In one embodiment, said body cavity is a nasal cavity. In one embodiment, said body cavity is the middle meatus. In one embodiment, said body cavity is the olfactory cleft. In one embodiment, said implant is non-metallic. In one embodiment, implant is placed in contact with a wet surface of a body cavity without a needle. In one embodiment, said implant lacks a retention frame.

The present invention in one embodiment contemplates a method of delivering an agent comprising: a) providing an implant comprising a plurality of fibers at least one said fibers comprising (i) a membrane wall containing at least one orifice, (ii) a lumen containing an agent, and (iii) a coating at least partially covering said membrane wall; and b) placing said implant in contact with a wet surface of a body cavity, such that said agent is delivered to said wet surface through osmosis. In one embodiment, said wet surface of a body cavity is within a human or animal body. In one embodiment, said fibers are bioresorbable. In one embodiment, said fibers are biodurable. In one embodiment, said membrane wall is water permeable. In one embodiment, said membrane wall is not permeable to said agent. In one embodiment, said agent is a drug. In one embodiment, said agent is a steroid. In one embodiment, said agent is a glucocorticoid. In one embodiment, said agent is mometasone furoate. In one embodiment, said agent is solid. In one embodiment, said agent is continuous throughout said lumen. In one embodiment, said agent is a hydrophilic or lipophilic molecule. In one embodiment, said agent is a small molecule or biomacromolecule. In one embodiment, said coating is bioresorbable. In one embodiment, said coating is biodurable. In one embodiment, said coating is covering said at least one orifice. In one embodiment, said coating is not covering said at least one orifice. In one embodiment, said method further comprises a topcoat at least partially covering said coating. In one embodiment, said plurality of fibers are in an arrangement selected from the group consisting of a braid, a spiral, a mesh, and a weave. In one embodiment, said lumen further comprises an osmogen. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said lumen further comprises a permeability enhancer. In one embodiment, said lumen further comprises at least one aiding agent selected from the group consisting of a wicking agent, a surfactant, a swelling agent, and a solubilizing agent. In one embodiment, said lumen further comprises a polymer fiber core. In one embodiment, agent is contained between said polymer fiber core and said membrane wall. In one embodiment, said polymer is a shape-memory polymer fiber. In one embodiment, said polymer is swellable. In one embodiment, said polymer is loosely crosslinked. In one embodiment, said implant is self-expanding. In one embodiment, said implant is expandable. In one embodiment, said implant is balloon expandable. In one embodiment, said implant is configured to self-expand in said body cavity. In one embodiment, said wet surface is the surface of mucosal tissue. In one embodiment, said mucosal tissue is tissue of the middle meatus. In one embodiment, said mucosal tissue is tissue of the olfactory cleft. In one embodiment, said body cavity is a nasal cavity. In one embodiment, said body cavity is the middle meatus. In one embodiment, said body cavity is the olfactory cleft. In one embodiment, said implant is non-metallic. In one embodiment, said implant is placed in contact with a wet surface of a body cavity without a needle. In one embodiment, said implant lacks a retention frame. In one embodiment, at least one fiber has a diameter between 100-500 µm.

In one embodiment, a method of manufacturing an implant is contemplated, comprising: a) providing a polymer and an agent; b) extruding said polymer and agent so as to create a plurality of fibers comprising a lumen, wherein said lumen comprises said agent; c) arranging said plurality of fibers into a structure. In one embodiment, said polymer is semi-permeable. In one embodiment, said arranging is braiding. In one embodiment, said agent is co-extruded with said polymer. In one embodiment, said method further comprises the step of mixing said polymer with a salt before step a). In one embodiment, said salt is inorganic salt. In one embodiment, said method further comprises the step of drilling orifices into said plurality of fibers. In one embodiment, said drilling is mechanical drilling or laser drilling. In one embodiment, said method further comprises the step of coating said structure with an elastomer. In one embodiment, said coating is conformal. In one embodiment, said method further comprises providing an osmogen, wherein said lumen of step b) further comprises said osmogen. In one embodiment, said osmogen is a salt or sugar. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said osmogen is a water-soluble organic polymer. In one embodiment, said osmogen is selected from the group consisting of hydroxy propyl methyl cellulose (HPMC), sodium carboxy methyl cellulose (Na CMC), polyethylene oxide (PEO), polyvinyl pyrrolidine (PVP), methyl cellulose (MC), and their combinations at any ratios. In one embodiment, said osmogen is a water-soluble amino acid. In one embodiment, said osmogen is selected from the group consisting of alanine, glycine, leucine, methionine, and their combinations at any ratios. In one embodiment, said structure is selected from the group consisting of a braided structure, a spiral structure, a weave structure, and a mesh structure.

The present invention in one embodiment contemplates a method of manufacturing an implant, comprising: a) providing a solid polymer core, a polymer and an agent; b) successively coating said solid polymer core with said agent to produce an agent-coated fiber; c) successively coating said first fiber with said polymer to produce a polymer-coated fiber; d) arranging a plurality of said polymer-coated fibers into a structure. In one embodiment, said polymer is semi-permeable. In one embodiment, said arranging is braiding. In one embodiment, said method further comprises the step of drilling orifices into said polymer-coated fiber. In one embodiment, wherein said drilling is mechanical drilling or laser drilling. In one embodiment, said method further comprises the step of coating said structure with an elastomer. In one embodiment, said coating is conformal. In one embodiment, said method further comprises providing the step of mixing said agent with an osmogen prior to step b). In one embodiment, said osmogen is a salt or sugar. In one embodiment, said salt is inorganic salt. In one embodiment, said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. In one embodiment, said structure is selected from the group consisting of a braided structure, a spiral structure, a weave structure, and a mesh structure.

The present invention in one embodiment contemplates an system comprising an implant having a plurality of fibers at least one of said fibers comprising (i) a membrane wall containing at least one orifice, and (ii) a lumen loaded with an agent and an osmogen, wherein said implant is configured to have a release rate that allows for release of drug for more than 12 weeks. In one embodiment, said implant further comprises a coating at least partially covering said membrane wall.

The present invention in one embodiment contemplates a system comprising an implant having a plurality of fibers at least one of said fibers comprising (i) a membrane wall containing at least one orifice, and (ii) a lumen loaded with an agent, wherein said implant is configured to have a release rate that allows for release of 20 to 80% of said drug during the first 12 weeks. In one embodiment, said implant further comprises a coating at least partially covering said membrane wall.

The present invention in one embodiment contemplates a system comprising an implant having a plurality of fibers at least one of said fibers comprising (i) a membrane wall containing at least one orifice, and (ii) a lumen loaded with an agent, wherein said implant is configured to have a release rate that allows for a substantially linear release between 1 and 12 weeks. In one embodiment, said implant further comprises a coating at least partially covering said membrane wall.

The present invention in one embodiment contemplates a system comprising an implant having a plurality of fibers at least one of said fibers comprising (i) a membrane wall containing at least one orifice, and (ii) a lumen loaded with an agent, wherein said implant is configured to have a release rate that allows for a substantially linear release that is exhibited in vitro in pH 7.4 PBS buffer containing 2% SDS at 37° C. In one embodiment, said implant further comprises a coating at least partially covering said membrane wall.

The present invention in one embodiment contemplates a system comprising an implant having a plurality of fibers at least one of said fibers comprising (i) a membrane wall containing at least one orifice, and (ii) a lumen loaded with an agent and an osmogen, wherein said implant is configured to have a release rate that allows for release of drug for more than 12 weeks. In one embodiment, said implant further comprises a coating at least partially covering said membrane wall.

The present invention in one embodiment contemplates a system comprising an implant having a plurality of fibers at least one of said fibers comprising (i) a membrane wall containing at least one orifice, and (ii) a lumen loaded with an agent and an osmogen, wherein said implant is configured to have a release rate that allows for release of 20 to 80% of said drug during the first 12 weeks. In one embodiment, said implant further comprises a coating at least partially covering said membrane wall.

The present invention in one embodiment contemplates a system comprising an implant having a plurality of fibers at least one of said fibers comprising (i) a membrane wall containing at least one orifice, and (ii) a lumen loaded with an agent and an osmogen, wherein said implant is configured to have a release rate that allows for a substantially linear release between 1 and 12 weeks. In one embodiment, said implant further comprises a coating at least partially covering said membrane wall.

The present invention in one embodiment contemplates a system comprising an implant having a plurality of fibers at least one of said fibers comprising (i) a membrane wall containing at least one orifice, and (ii) a lumen loaded with an agent and an osmogen, wherein said implant is configured to have a release rate that allows for a substantially linear release that is exhibited in vitro in pH 7.4 PBS buffer containing 2% SDS at 37° C. In one embodiment, said implant further comprises a coating at least partially covering said membrane wall.

The present invention further contemplates an embodiment comprising a method of delivering an agent comprising: a) providing an expandable implant comprising (i) at least one membrane wall containing at least one orifice, (ii) a lumen containing an agent, and (iii) a coating at least partially covering said membrane wall; and b) placing said implant in contact with mucosal tissue such that at least a portion of said agent is delivered to said mucosal tissue through osmosis. In one embodiment, said mucosal tissue is within a human or animal body. In one embodiment, said mucosal tissue is selected from the group consisting of nose, gut, and lung tissue. In one embodiment, said mucosal tissue is selected from the group consisting of tissues of the oral cavity pharynx, tonsils, urethra, and vagina. In one embodiment, said mucosal tissue is the middle meatus. In one embodiment, said mucosal tissue is tissue of the olfactory cleft. In one embodiment, said lumen further comprises an osmogen. In one embodiment, said coating is elastomeric. In one embodiment, said membrane wall is water permeable. In one embodiment, said membrane wall is not permeable to said agent. In one embodiment, said agent is a therapeutic agent. In one embodiment, said agent is a steroid. In one embodiment, said agent is a glucocorticoid. In one embodiment, said agent is mometasone furoate. In one embodiment, said agent is a protein or peptide. In one embodiment, said agent is an antibody. In one embodiment, said coating is covering said at least one orifice. In one embodiment, said coating is not covering said at least one orifice. In one embodiment, the implant further comprises a topcoat at least partially covering said coating. In one embodiment, said lumen further comprises a polymer core. In one embodiment, said agent is contained between said polymer core and said membrane wall.

In yet another embodiment, the present invention contemplates an implant comprising a plurality of fibers at least one of said fibers comprising (i) a membrane wall containing at least one orifice, (ii) a lumen containing an agent, and (iii) a coating at least partially covering said membrane wall. In one embodiment, said implant is expandable. In one embodiment, said agent is a drug. In one embodiment, said agent is a steroid. In one embodiment, said agent is a glucocorticoid. In one embodiment, said agent is mometasone furoate. In one embodiment, said agent is a peptide or protein. In one embodiment, said agent is an antibody. In one embodiment, said lumen further comprises an osmogen.

In yet another embodiment, the present invention contemplates an expandable implant comprising a plurality of fibers at least one of said fibers comprising (i) at least one membrane wall containing at least one orifice, (ii) a lumen containing an agent, and (iii) a coating at least partially covering said membrane wall. In one embodiment, said lumen further comprises an osmogen. In one embodiment, said agent is mometasone furoate. In one embodiment, said agent is a peptide or protein. In one embodiment, said agent is an antibody.

In still another embodiment, the present invention contemplates an implant comprising (i) a first membrane layer containing at least one orifice, (ii) a second membrane layer, and (iii) an active layer containing an agent and an osmogen disposed between said first and second membrane layers. In one embodiment said agent is a drug. In one embodiment, said agent is a steroid. In one embodiment, said agent is a glucocorticoid. In one embodiment said agent is mometasone furoate. In one embodiment, said agent is solid. In one embodiment, said agent is a small molecule or biomacromolecule. In one embodiment, said agent is a peptide or protein.

### DEFINITIONS

The term “implant” as used herein, relates to a device or system to be inserted into tissue, organ, or part of the body or introduced into a bodily cavity. As used herein, “device,” “scaffold,” “stent”, “carrier”, “matrix”, and “implant” may be used synonymously.

The term “scaffold” as used herein, relates to a structure comprising a supporting framework. For example, a device comprising a structure of fibers.

The term “braided” as used herein, relates to a structure, such as a device, comprising one or more intertwined strands.

The term “helical” as used herein, relates to a spiral or helical shaped structure, comprising one or more strands. As used herein, “helical” and “spiral” may be used synonymously.

The term “spiral” as used herein, relates to a spiral or helical shape structure, comprising one or more strands. As used herein, “helical” and “spiral” may be used synonymously.

The term “mesh” as used herein, relates to a structure, such as a device, made out of a network of fibers.

The tern “weave” as used herein, relates to a structure, such as a device, made out of interlaced fibers passing in on direction with others at a right angle to them.

The term “tubular” as used herein, relates to hollow shapes of circular cross-section or non-circular cross-section (e.g., oval, etc.) and hollow shapes of constant diameter or variable diameter (e.g. tapered diameter, such as in a hollow frustum). Both ends of the generally tubular scaffold may be open, one end may be open and the other end closed, or both ends may be closed.

The term “expandable” as used herein, relates to a structure that has the ability to expand or widen. The term “self-expanding” as used herein, relates to the ability for a device to expand or widen after having been contracted. The term “self-expanding” is intended to include devices that are crimped to a reduced configuration for delivery into the body, and thereafter are able expand to a larger suitable configuration (i.e. larger than the crimped configuration) once released from the delivery configuration, either without the aid of any additional expansion devices or with the partial aid of balloon-assisted or similarly-assisted expansion.

The term “osmosis” as used herein, relates to passage of fluid or molecules through a semi-permeable or permeable material. A non-limiting example includes movement of a solvent across a semipermeable membrane toward a higher concentration of solute.

The term “osmotic pump” as used herein, relates to delivery systems using movement across a permeable or semi-permeable material.

The term “osmogen” as used herein, relates to agents used to enhance osmosis.

The term “permeable” as used herein, relates to a material which allows fluids or molecules to pass through.

The term “semi-permeable” as used herein, relates to a material of which at least a portion allows fluids or molecules to pass through.

The term “strands,” “filaments,” and “fibers” may be used interchangeably and include single strands, filaments, and fibers, as well as multi-fiber strands and filaments.

The term “sheet” as used herein, relates to flat devices and systems.

The term “orifice” as used herein, relates to holes or openings within devices and systems.

The term “lumen” as used herein, relates to hollow spaces within bodily systems, devices, etc.

The term “rolled” as used herein, relates to a material wrapping around a hollow space or around itself.

The term “drug delivery” as used herein, relates to systems for transporting pharmaceutical compounds to a bodily system.

The term “drug” as used herein, relates to a pharmaceutical compound.

The term “active pharmaceutical ingredient” as used herein, relates to a substance or mixture of substances that are intended to furnish pharmacological activity or other effect in the diagnosis, cure, mitigation, treatment, or prevention of disease or to affect the structure or function of the body.

The term “agent” as used herein, relates to a substance that brings about a chemical, biological, or physical effect or reaction.

The term “therapeutic agent” as used herein, relates to a substance or compound (drug, protein, peptide, gene, etc.) capable of having a healing or treating effect.

The term “substance” as used herein, relates to a physical matter.

The term “small molecule” refers to a molecule below the molecular weight of 1 kDa.

The term “biomacromolecule” as used herein, relates to biomolecules having a molecular weight over 0.8 kDa.

The term “coating” as used herein, relates to a layer. The terms “coating” and “covering” are used synonymously herein.

The term “membrane” as used herein, relates to barrier or lining. It can be a selective barrier, allowing some things to pass through but stopping others. Such things may be molecules, ions, or other small particles.

The term “biodegradable” as used herein, relates to the ability to degrade in a bodily system. The term “bioresorbable” as used herein, relates to the ability to degrade in a bodily system. As used herein, “biodegradable” and “bioresorbable” may be used synonymously.

The terms “nonbiodegradable” and “biodurable” as used herein, relate to the ability to not degrade in a bodily system. As used herein, “nonbiodegradable” and “biodurable” may be used synonymously.

The term “aiding agent” as used herein, relates to substances which may be added to a system to aid its use.

The term “wicking agent” as used herein, relates to substances which may aid in the ability to absorb or draw in fluid or molecules.

The term “swelling agent” as used herein, relates to relates substances which may aid in the ability for a material to swell or enlarge.

The term “surfactant” as used herein, relates to substances which tends to reduce the surface tension of a fluid in which it is added.

The term “solubilizing agent” as used herein, relates to a substance which may increase solubility of one substance in another.

The term “permeability enhancer” as used herein relates to a substance which serves to facilitate the permeability of the drug into tissues or across tissue boundaries, such as the blood brain barrier for example.

The term “polymer” as used herein, relates to a substance which has a molecular structure consisting partly or entirely of a large number of units bonded together.

The term “hydrophilic” as used herein, relates to an ability to at least partially dissolve or be wetted by water. A hydrophilic molecule or portion of a molecule is one whose interactions with water and other polar substances are more thermodynamically favorable than their interactions with oil or other hydrophobic solvents. They are typically charge-polarized and capable of hydrogen bonding.

The term “lipophilic” as used herein, relates to an ability to at least partially repel water, or relates to an ability to at least partially dissolve in lipids or fats. As used herein, “lipophilic” and “hydrophobic” may be used synonymously.

The term “cavity” as used herein, relates to an empty space within an object, such as within a human or animal body.

The terms “mucosal tissue” and “mucosal surface” are meant to indicate the surface areas that comprise the mucosa. The mucosa is characterized by the presence of a semipermeable epithelial barrier. Mucosal tissue surfaces are characterized by the presence of an overlying mucosal fluid (making them typically a wet surface), for example fluids such as saliva, tears, nasal, gastric, cervical and bronchial mucus. Thus, such surfaces are found in the eyes, nose, gut, and lung. Additional mucosal surfaces are found in the oral cavity (e.g. the mouth), pharynx, tonsils, urethra, and vagina.

The term “nasal cavity” as used herein, relates to the space, cavity, or lumen above and behind the nose in the middle of a face. Lumens in the nasal cavity include the superior meatus, the middle meatus, and the inferior meatus. Another nasal cavity is the “olfactory cleft.” The olfactory cleft refers to a paired orifice located in the medial and upper regions of the nasal cavity. This cleft is limited by the middle turbinate laterally, the nasal septum medially, the cribriform plate and the superior turbinate superiorly, the inferior margin of the middle turbinate inferiorly, and the anterior face of sphenoid sinus posteriorly.

The term “sinus” as used herein, relates to the paranasal sinuses, the spaces, cavities, or lumens in the cranial bones. Sinus cavities include the frontal sinus, the sphenoid sinus, the ethmoid air cells, and the maxillary sinus.

The term “sinus condition” as used herein, relates to an illness of the sinus and sinus cavities. The term “chronic” as used herein, relates to persisting or recurring illness or symptoms.

The “core-shell structure” as used herein, relates to a structure comprising multiple layers or “shells,” wherein the innermost layer may be called a “core.”

### BRIEF DESCRIPTION OF FIGURES

FIG. 1 shows a schematic illustration of a self-expandable implant comprising osmotic drug delivery fibers (100) either not comprising orifices or comprising orifices under an opaque coating.

FIG. 2 shows a schematic illustration of an osmotic drug delivery fiber embodiment comprising one or more delivery orifices (200), a semi-permeable polymer membrane (201), an API (202) and an osmogen (203).

FIG. 3 shows a schematic illustration of an osmotic drug delivery fiber embodiment comprising one or more delivery orifices (300), a semi-permeable polymer membrane (301), an API (302), a polymer fiber core (303) and an osmogen (304).

FIG. 4 shows a schematic illustration of a spiral scaffold embodiment for osmotic drug delivery comprising a spiral scaffold (400) with a delivery orifice (401), with an expanded end-view showing the semi-permeable polymer membrane (402), an API (403), and an osmogen (404).

FIG. 5 shows a schematic illustration of a rolled osmotic drug delivery sheet embodiment comprising one or more delivery orifices (500), a semi-permeable polymer membrane (501), an API (502) and an osmogen (503).

FIG. 6 shows a schematic illustration of a self-expandable implant embodiment comprising osmotic drug delivery fibers (600) comprising orifices (601).

FIG. 7 shows nasal cavity structures including the olfactory cleft.

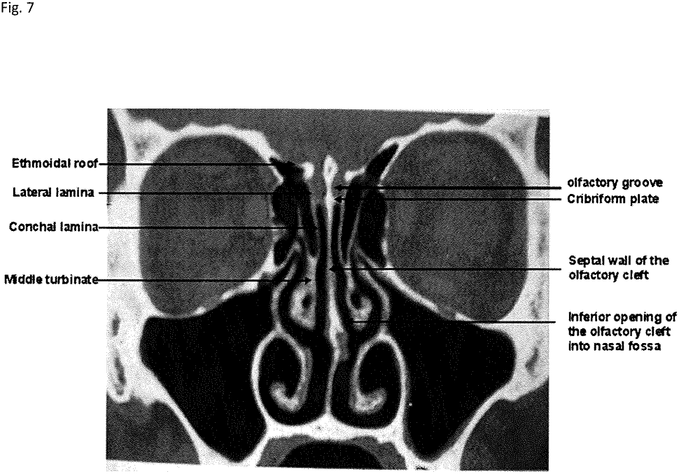

### DESCRIPTION OF THE INVENTION

One exemplary embodiment of the present implantable devices is a device comprising of one or more fibers, at least one of which is a permeable, hollow fiber comprising an agent or active ingredient. This device, or scaffold, is not limited to the number of fibers or structure the fibers take. Another exemplary embodiment of the present implantable devices is a device comprising a permeable or semi-permeable, sheet, which contains an active ingredient. The fiber or sheet may be considered a permeable or semi-permeable membrane.

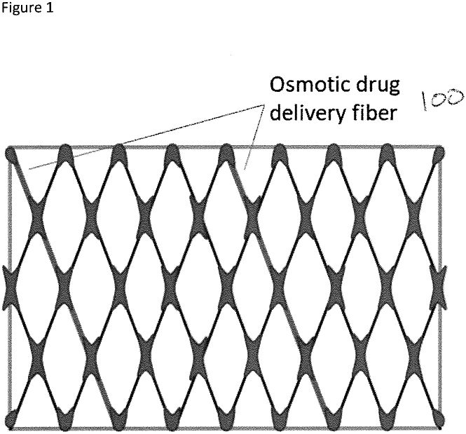

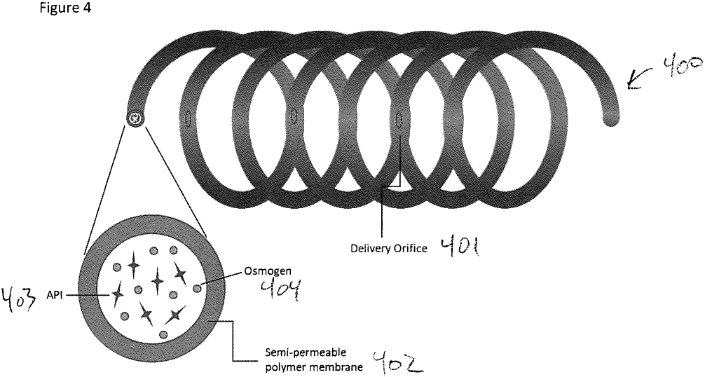

The embodiment of the device comprising one or more fibers contains osmotic drug delivery components. In this exemplary embodiment, drug delivery components are comprised of one or more permeable or semi-permeable polymeric, hollow fibers filled with a drug or active pharmaceutical ingredient (API) in the absence or presence of osmogens. The present invention is not limited by the number or arrangement of the fiber(s). In one embodiment, fiber arrangement is a spiral, as seen in FIG. 4. One embodiment, the fiber arrangement is braided. In this design, shown in FIG. 1, the implants comprise a fiber-based braid structure with multiple strands (e.g. 2 to 64), where at least one fiber comprises semi-permeable membrane that encapsulates the API(s).

The embodiment of the device comprising a sheet may contain osmotic drug delivery components. The permeable or semi-permeable sheets may be implanted flat or in a rolled state. In the rolled embodiment, the rolled sheet comprises an internal lumen. In this exemplary embodiment, drug delivery components are comprised of a semi-permeable polymeric hollow sheet filled with a drug or active pharmaceutical ingredient (API) in the absence or presence of an osmogen.

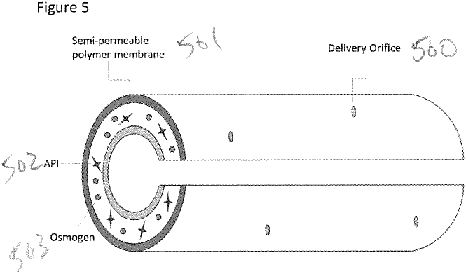

The implantable device may comprise a permeable or semi-permeable membrane, such as one or more fibers or a sheet, as seen in FIG. 5. In one embodiment, permeability to fluid is achieved through the use of permeable materials. In another embodiment, permeability is achieved through one or more delivery orifices on the hollow fiber or sheet wall. Any number of orifices is contemplated, including, but not limited to, one, two, three, four, five, six, seven, eight, nine, ten, twenty-five, fifty, one hundred, two hundred, a thousand, etc.

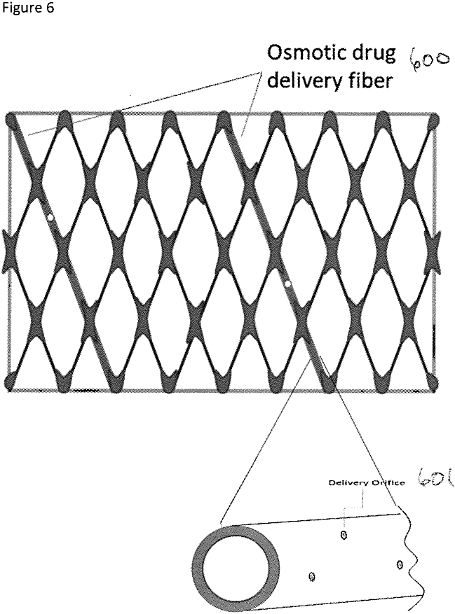

In one embodiment, the devices herein may be coated or covered. It is not intended for the present invention to be limited by the type, thickness, or coverage of the coating, such as an elastomer. The device may be completely or partially coated. In one embodiment, there may be an elastomer coating on the top of the permeable or semi-permeable membrane, such as the hollow fibers or sheet, covering or not covering any delivery orifices, as seen in FIG. 6. Elastomers may be coated onto the implants to provide them with self-expandability. One or more orifices may be formed on the semi-permeable membrane either before or after the elastomer coating.

In one embodiment, the device may be expandable. In one embodiment, the device may be self-expanding. In one embodiment, the device may be balloon-expandable. The many scaffold embodiments of the present disclosure may be self-expanding in that they are manufactured at a first diameter, subsequently reduced or “crimped” to a second, reduced diameter for placement within a delivery catheter, and self-expand towards the first diameter when extruded from the delivery catheter at an implantation site. The first diameter may be at least 10% larger than the diameter of the bodily lumen into which it is implanted in some embodiments. The scaffold may be designed to recover at least about 70%, at least about 80%, at least about 90%, up to about 100% of its manufactured, first diameter, in some embodiments.

In one embodiment, the device may be biodegradable or biodurable.

In one embodiment, various components of the device may be hydrophilic, hydrophobic, lipophilic, etc.

Upon implantation, a fluid, such as water, enters the lumen through the permeable or semi-permeable wall, forming an osmotic pressure gradient that pushes the active pharmaceutical ingredient (API) out of the delivery orifices at a steady rate. These osmotic dosage forms function by allowing a fluid, such as water, around the implant to flow through the semi-permeable membrane, dissolve the API in the core so it can be released through the ports in the membrane by the osmotic pressure.

The present devices and systems may be used with a large multitude of active ingredients. Agents, such as active pharmaceutical ingredients (APIs), may be embedded in porous or semi-porous fiber strands or sandwiched in porous or semi-porous sheets. In one embodiment, the agent is an active pharmaceutical ingredient. In one embodiment the present agent is a therapeutic agent. In one embodiment, the present agent is a glucocorticoid. In one embodiment, the present agent is mometasone furoate.

The polymers used in the implants can be biodegradable, nonbiodegradable or biodurable. Polymers used in the implantable device include cellulose esters, alkyl-celluloses, and cellulose derivatives including methylcellulose, ethyl cellulose, hydroxyethyl cellulose, hydroxypropyl cellulose, hydroxylpropyl methyl cellulose, cellulose nitrate, cellulose acetate ethyl carbamate, cellulose acetate phthalate, cellulose acetate dimethaminoacetate, cellulose acetate ethyl carbonate, cellulose acetate chloroacetate, cellulose acetate ethyl oxalate, or any combination of any thereof. Synthetic polymers that may be used in the present device include partially and completely hydrolyzed alkylene-vinyl acetate copolymers, hydroxylated and unhydroxylated ethylene-vinyl acetate copolymers, derivatives of polystyrene such as poly(sodium styrenesulfonate) and poly(vinylbenzyltrimethylammonium chloride), homo- and copolymers of polyvinyl acetate, polymers of acrylic acid and methacrylic acid, copolymers of an alkylene oxide and alkyl glycidyl ether, polyurethanes, polyamide, polyshulphones, crosslinked poly(ethylene oxide), poly(alkylenes), poly(vinyl imidazole). Semi-permeable bioresorbable polymers that may be used in the present device include polyglycolic acid, polylactic acid, polycaprolactone, polydioxanone, poly(trimethylene carbonate), poly(3-hydroxybutyrate), poly(propiolactone), polyethylene succinate), poly(butylenes succinate), poly(3-hydroxybutyrate-co-3-hydroxyvalerate), poly(ester carbonate), poly(glycerol sebacate), and their copolymers and derivatives thereof.

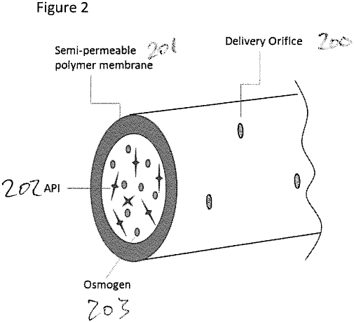

The device may comprise a variety of substances, as seen in FIG. 2, such as the API, osmogens, and other aiding agents (e.g. wicking agents, surfactants, and solubilizing agents), while the shell is comprised of the semi-permeable polymer. Various osmogens, or osmotic agents, can be used to tailor the osmotic pressure inside the semi-permeable membrane and consequently the drug release rate. These osmogens include but not limited to sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. The osmogen can also be a water-soluble organic polymer such as hydroxy propyl methyl cellulose (HPMC), sodium carboxy methyl cellulose (Na CMC), polyethylene oxide (PEO), polyvinyl pyrrolidine (PVP), and methyl cellulose (MC) or a water-soluble amino acid such as alanine, glycine, leucine, and methionine.

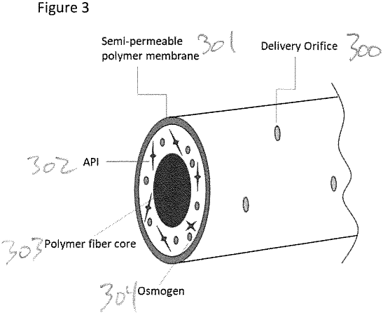

The present invention is advantageous as it can be formed through a variety of manufacturing methods, such as coextrusion, filling, or successive coating. In one embodiment, drug-encapsulated fibers are formed by coextrusion of the API(s) and the semi-permeable polymers into a core-shell structure of extrusion. In another embodiment, drug-encapsulated fibers are formed by first hollow fibers comprising a lumen, followed by filling the lumen with API(s). As seen in FIG. 2, the lumen of the osmotic drug delivery fiber may comprise a variety of substances, such as the API, osmogens, and other aiding agents (e.g. wicking agents, surfactants, and solubilizing agents), while the shell may be comprised of the semi-permeable polymer. In another embodiment, the drug-encapsulated fibers are formed by coating a solid polymer fiber core successively with the API(s) and a semi-permeable polymer membrane. As shown in FIG. 3, the APIs are encapsulated in between the polymer fiber core and the semi-permeable polymer membrane to form a sandwich structure. The two ends of the fiber comprising API may be blocked through polymer coating or welding. Following the fabrication of the individual fibers, one or more fibers, some containing API and some not, can be formed into an implant or scaffold.

One or more fibers described above can be fabricated into spiral scaffolds, with at least one comprising API, as seen in FIG. 4. Following the blockage of the ends of the fibers, one or more drug delivery orifices may be placed on the semi-permeable wall using, for example, laser drilling. A shape memory polymer fiber may be attached to the side or even serve as the core of the drug delivery fiber, to maintain the spiral shape of the fiber after implantation. In addition, an elastomer can be coated onto the spiral scaffolds to enhance their recoverability post implantation.

Multi-stranded scaffolds comprising other fiber arrangements are manufactured following fabrication of single fibers, comprising at least one strand of those API-encapsulated fibers. The fibers may be arranged in a spiral, as stated above, or a braid, mesh, etc. The scaffolds can be conformally coated with an elastomer to provide the scaffold self-expandability. Following the blockage of the ends of the fibers, one or more drug delivery orifices may be placed on the semi-permeable wall using, for example, laser drilling.

Before or after such elastomer coating or arrangement of fibers, one or more delivery orifices are introduced onto the semi-permeable membrane of each API-encapsulated fiber through either mechanical drilling or laser drilling. Either luminal or abluminal delivery orifices can be formed accordingly. The size, density, and location of the delivery orifices are determined by the API used, the target implantation sites, and the dosing requirement. Furthermore, the delivery orifices can also be formed by a salt-leaching approach, where inorganic salt granules are present during the semi-permeable membrane formation. Upon implantation, the salt will dissolve and leach out to form drug delivery orifices in situ. The number and size of the orifices can be tuned by tailoring the size and quantity of salt granules within the membrane.

The sheet embodiment also may be manufactured in a variety of methods. APIs and aiding agents are encapsulated in between two polymer membranes to form a drug release sheet. One or both polymer membranes are semi-permeable membranes. Drug delivery orifices can be drilled on either polymer membranes to allow drug release. Optional elastomer coating can be further introduced onto the rolled sheets to improve their self-expandability.

1. A method of delivering an agent comprising:
a) providing an expandable implant comprising (i) at least one membrane wall containing at least one delivery orifice, (ii) a lumen containing an agent, and (iii) a coating at least partially covering said membrane wall; and
b) placing said implant in contact with mucosal tissue of the middle meatus such that at least a portion of said agent is delivered to said mucosal tissue through osmosis.

2-5. (canceled)

6. The method of claim 1, wherein said lumen further comprises an osmogen.

7. The method of claim 1, wherein said coating is elastomeric.

8. The method of claim 1, wherein said membrane wall is water permeable.

9. The method of claim 1, wherein said membrane wall is not permeable to said agent.

10. The method of claim 1, wherein said agent is a therapeutic agent.

11. The method of claim 1, wherein said agent is a steroid.

12. The method of claim 1, wherein said agent is a glucocorticoid.

13. The method of claim 1, wherein said agent is mometasone furoate.

14. The method of claim 1, wherein said agent is a protein or peptide.

15. The method of claim 1, wherein said agent is an antibody.

16. The method of claim 1, wherein said coating is covering said at least one orifice.

17. The method of claim 1, wherein said coating is not covering said at least one orifice.

18. The method of claims 1, wherein said implant further comprises a topcoat at least partially covering said coating.

19. The method of claim 1, wherein said lumen further comprises a polymer core.

20. The method of claim 19, wherein said agent is contained between said polymer core and said membrane wall.

21. An implant comprising a plurality of fibers at least one of said fibers comprising (i) a membrane wall containing at least one delivery orifice, (ii) a lumen containing mometasone furoate, and (iii) a coating at least partially covering said membrane wall.

22. The implant of claim 21, wherein said implant is expandable.

23-28. (canceled)

29. The implant of claim 21, wherein said lumen further comprises an osmogen.

30. An expandable implant comprising a plurality of fibers at least one of said fibers comprising (i) at least one membrane wall containing at least one delivery orifice, (ii) a lumen containing an agent and an osmogen, and (iii) a coating at least partially covering said membrane wall.

31. The implant of claim 30, wherein said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations.

32. The implant of claim 30, wherein said agent is mometasone furoate.

33. The implant of claim 30, wherein said agent is a peptide or protein.

34. The implant of claim 30, wherein said agent is an antibody.

35. A implant comprising (i) a first membrane layer containing at least one orifice, (ii) a second membrane layer, and (iii) an active layer containing mometasone furoate and an osmogen disposed between said first and second membrane layers.

36-38. (canceled)

39. The implant of claim 35, wherein said osmogen is selected from the group consisting of sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations.

40. The implant of claim 35, wherein said osmogen is selected from the group consisting of alanine, glycine, leucine, and methionine.

41-42. (canceled)

43. A method of delivering an agent comprising:
a) providing an expandable implant comprising (i) at least one membrane wall containing at least one delivery orifice, (ii) a lumen containing an agent and an osmogen, and (iii) a coating at least partially covering said membrane wall; and
b) placing said implant in contact with mucosal tissue of the middle meatus such that at least a portion of said agent is delivered to said mucosal tissue through osmosis.

44. The method of claim 43, wherein said membrane wall is water permeable.

45. The method of claim 43, wherein said membrane wall is not permeable to said agent.

46. The method of claim 43, wherein said agent is a therapeutic agent.

47. The method of claim 43, wherein said agent is a steroid.

48. The method of claim 43, wherein said agent is a glucocorticoid.

49. The method of claim 43, wherein said agent is mometasone furoate.

50. The method of claim 43, wherein said agent is a protein or peptide.

51. The method of claim 43, wherein said agent is an antibody.

52. The method of claim 43, wherein said coating is covering said at least one orifice.

53. The method of claim 43, wherein said coating is not covering said at least one orifice.
